(19) United States
(12) Patent Application Publication
(10) Pub. No.: US 2023/0218371 A1
(43) Pub. Date: Jul. 13, 2023

(54) DISPLAY OF MULTIPLE AUTOMATED ORTHODONTIC TREATMENT OPTIONS

(71) Applicant: 3M INNOVATIVE PROPERTIES COMPANY, St. Paul, MN (US)

(72) Inventors: Alexandra R. Cunliffe, St. Paul, MN (US); Elisa J. Collins, Oakdale, MN (US); Guruprasad Somasundaram, St. Paul, MN (US); Nitsan Ben-Gal Nguyen, Apple Valley, MN (US); Benjamin D. Zimmer, Hudson, WI (US); Cody J. Olson, Ham Lake, MN (US)

(21) Appl. No.: 17/928,022

(22) PCT Filed: May 11, 2021
(86) PCT No.: PCT/IB2021/054027

§ 371 Date: Nov. 28, 2022

Related U.S. Application Data
(60) Provisional application No. 63/033,881, filed on Jun. 3, 2020.

(51) Int. Cl. A61C 7/00; A61C 13/34

(52) U.S. Cl. CPC A61C 7/002; A61C 13/34

(57) ABSTRACT

Methods for generating multiple orthodontic treatment options for a digital 3D model of teeth in malocclusion. The method generates a plurality of different orthodontic treatment plans for the teeth and displays in a user interface the digital 3D model of teeth in malocclusion with a visual indication of each of the plurality of different orthodontic treatment plans. The visual indication of the treatment plans can be overlaid on the digital 3D model of teeth in malocclusion and possibly include aligners, brackets, or a combination of aligners and brackets. A doctor, technician, or other user can then select one of the treatment plans for a particular patient.

### BACKGROUND

The goal of the orthodontic treatment planning process is to determine where the post-treatment positions of a person's teeth (setup state) should be, given the pre-treatment positions of the teeth in a malocclusion state. This process is typically performed manually using interactive software and is a very time-consuming process. A need thus exists for an algorithm to display and evaluate treatment options.

### SUMMARY

A computer-implemented method for generating multiple orthodontic treatment options includes receiving a digital 3D model of teeth in malocclusion and generating a plurality of different orthodontic treatment plans for the teeth. The method displays a visual indication of each of the plurality of different orthodontic treatment plans for possible selection.

A computer-implemented method for displaying a user interface comprising orthodontic treatment options includes a visual indication of different orthodontic treatment plans with or without malocclusion overlays and optionally with a separate display of a digital 3D model of teeth in malocclusion corresponding with the treatment plans.

### BRIEF DESCRIPTION OF THE DRAWINGS

FIG. 1 is a diagram of a system for receiving and processing digital models based upon 3D scans.

FIG. 2 is a flow chart of a method to generate and display multiple orthodontic treatment options.

FIG. 3 is a user interface to automatically display options for orthodontic treatment plans.

FIG. 4 is a user interface to display a malocclusion and versions of a setup for treatment of the malocclusion.

FIG. 5 is a user interface to display a malocclusion and multiple orthodontic treatment options customized for particular preferences.

### DETAILED DESCRIPTION

### Overview

Embodiments include a system to display multiple orthodontic treatment options to a user (e.g., doctor, technician, or patient). A digital three-dimensional (3D) model of a patient's malocclusion is received input. Optional input includes one or more treatment guidelines, for example whether to apply IPR (interproximal reduction, also known as teeth shaving), duration of treatment, or others. Based on the input, the system generates several candidates for a treatment plan. These candidate treatments may be generated based on factors including user preferences, treatment appliances, treatment strategy, or types of interventions or features (e.g., IPR or attachments). The user can then select one of the candidate treatment plans from the set of treatment plan options and either make additional refinements to the selected treatment plan, begin the aligner tray manufacturing process directly from this output, or send the selected treatment plan to a lab or other facility for further refinement by a technician. Generating and displaying multiple orthodontic treatment options to a practitioner could enable greater choice, flexibility, and control by the doctor and patient, or other users.

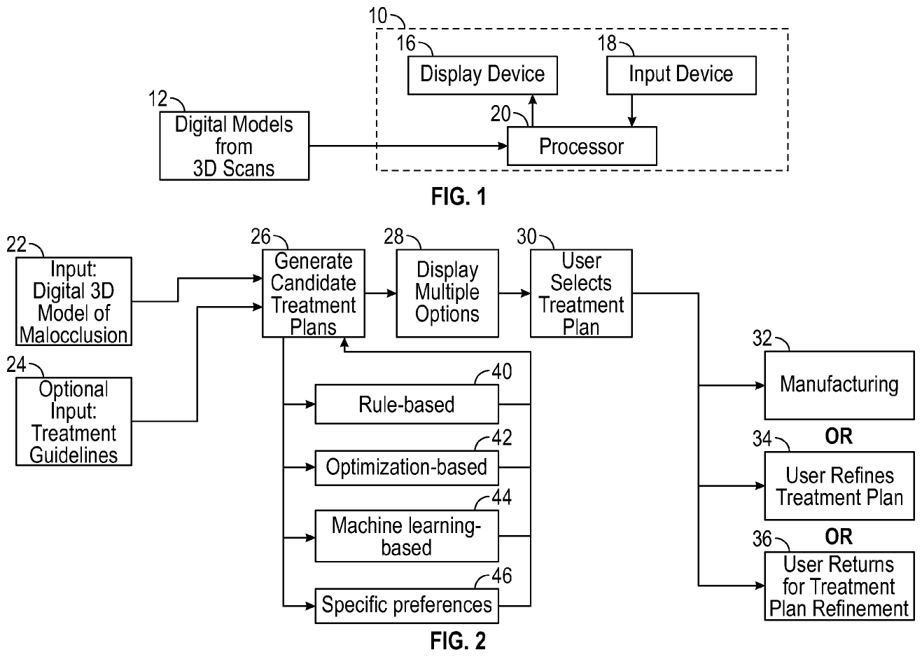

FIG. 1 is a diagram of a system 10 for receiving and processing digital 3D models based upon intra-oral 3D scans. System 10 includes a processor 20 receiving digital 3D models of teeth (12) from intra-oral 3D scans or scans of impressions of teeth. System 10 can also include an electronic display device 16, such as a liquid crystal display (LCD) device, and an input device 18 for receiving user commands or other information. Systems to generate digital 3D images or models based upon image sets from multiple views are disclosed in U.S. Pat. Nos. 7,956,862 and 7,605,817, both of which are incorporated herein by reference as if fully set forth. These systems can use an intra-oral scanner to obtain digital images from multiple views of teeth or other intra-oral structures, and those digital images are processed to generate a digital 3D model representing the scanned teeth. System 10 can be implemented with, for example, a desktop, notebook, or tablet computer. System 10 can receive the 3D scans locally or remotely via a network.

### Display of Multiple Treatment Options

The system receives a patient's malocclusion scan as input and displays multiple orthodontic treatment options to a user based on the input scan. Optionally, a set of doctor, technician, or patient preferences can also be included as input. For example, a doctor may wish to only see treatment options that do not require IPR, or only see treatment options that will take less than six months to complete.

Based on the inputs, a set of potential treatment plans can be displayed to the user. Some possibilities for treatment options include:

1. Appliances, for example dual arch aligners, brackets on one or both arches, brackets followed by aligners.

2. Treatment duration, for example six month treatment or two year treatment.

3. Treatment strategies, for example expansion, bite closure, or others.

4. Aligner features or lack thereof, for example attachments versus no attachments.

5. Doctor-specific treatment strategies, for example characteristic setups from a group of doctors.

6. Treatment finishing approaches, for example overcorrection.

7. Treatment interventions, for example IPR.

8. Doctor- or technician-specific preferences.

9. Patient-specific preferences, for example decreased pain (which could then increase the time of treatment) or lowered cost of treatment.

FIG. 2 is a flow chart of a method to generate and display multiple orthodontic treatment options. This method can be implemented in software or firmware for execution by processor 20. This method receives as input a digital 3D model of malocclusion (step 22) and optionally treatment guidelines (step 24). The method generates candidate treatment plans (step 26), for example using a rule-based approach (step 40), an optimization-based approach (step 42), a machine learning-based approach (step 44), or specific preferences (step 46). The method displays the multiple options for treatment plans on an electronic display device (step 28) and allows a user to select one of the treatment plans (step 30). After the user selects a treatment plan, the method can involve manufacturing appliances for the treatment plan (step 32), receiving a user refinement of the treatment plan (step 34), or the user returning the treatment plan for refinement (step 36).

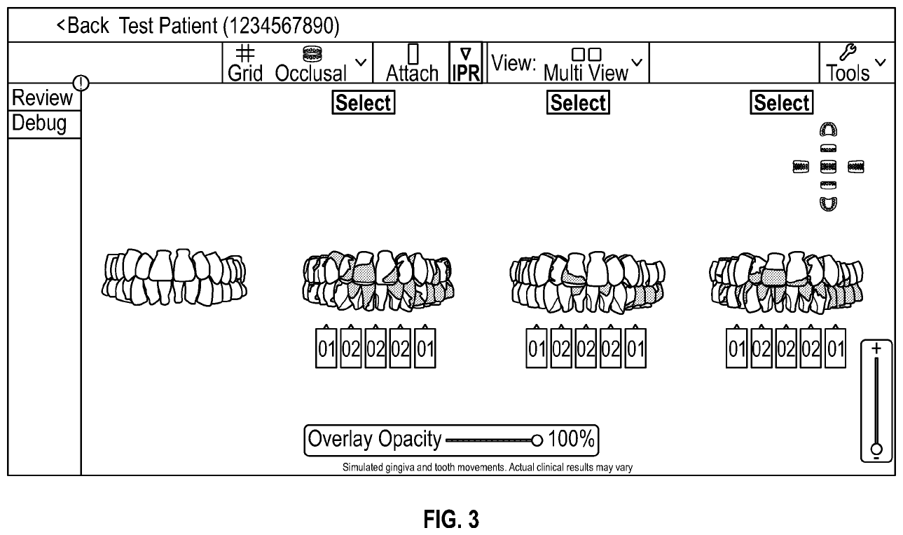

FIG. 3 is a user interface to automatically display options for orthodontic treatment plans, displaying the digital 3D model of teeth in malocclusion shown separately (left image) and three treatment plan options for this case (second, third, and fourth images from the left). The user may select a treatment from these three displayed options. The shading on the digital 3D models of teeth in the second, third, and fourth images represents the overlay between the malocclusion and the final state of the teeth (“malocclusion overlays”). The numbers below the digital 3D models of teeth represent the amount of IPR for each corresponding tooth. The user interface in FIG. 3 can be displayed on electronic display device 16. Also, the user interface can include user interactive tools or commands to vary the overlay opacity, zoom in or zoom out, and select a particular view of the digital 3D models of teeth for display (e.g., front, top, or side views).

The overlay opacity tool can also be used to eliminate the shading in the view or show full shading on the digital 3D model of teeth. By varying the shading in such manner and selectively displaying the malocclusion separately, the user interface can be configured to display one or more of the following: the digital 3D model of teeth in malocclusion shown separately with malocclusion overlays; only the malocclusion overlays of the treatment plans without the digital 3D model of teeth in malocclusion shown separately; the digital 3D model of teeth in malocclusion shown separately with the treatment plans and without the malocclusion overlays; and the treatment plans without the digital 3D model of teeth in malocclusion shown separately and without the malocclusion overlays.

The user interface in FIG. 3 shows three treatment plans for exemplary purposes; more or fewer treatment plans can be generated and shown in the same or a similar manner.

### Automatically Create Treatment Options to Display

There are several ways to use a computerized system to learn multiple treatment options that can be displayed.

### 1. Rule-Based Approach (Step 40).

Final setups can be automatically created such that they adhere to a set of rules. For example:

Treatment strategies, for example expansion—move the molars in the positive direction normal to the arch form between the malocclusion and the setup.

Treatment interventions, for example IPR—expand the front teeth between malocclusion and setup to make space and do not apply any IPR in the setup, or, do not expand the front teeth and apply IPR.

An example of a rule-based approach to generate setups is disclosed in PCT Patent Application Publication No. WO 2019/069191, which is incorporated herein by reference as if fully set forth.

### 2. Optimization-Based Approach (Step 42).

One optimization algorithm that creates final setups based on optimizing a set of metrics subject to some constraints in described in PCT Patent Application Publication No. WO 2020/026117, which is incorporated herein by reference as if fully set forth. Table 1 provides exemplary pseudocode for generating final setups for this optimization-based approach.

TABLE 1
Algorithm for optimization-based final setup generation

```
Given a state (arrangement of digital teeth in the mouth) and a required maximum score
(MaxScore):
Score = ScoringFunction(state) # compute score of current state
If (Score > MaxScore): # stopping criteria, exit routine once Score is less than or equal to MaxScore
  NewState = Perturb(State) # apply a Perturb function to move one or more teeth in the state
  NewState = Constrain(NewState) # apply a Constrain function to adjust tooth positions so that
  they meet constraints
  NewStateScore = ScoringFunction(NewState)
  If (NewStateScore < Score): # check if score has improved
    State = NewState
Constrain function = operation performed on state to ensure state meets requirements (e.g. tooth
movement limits)
Scoring function:
$Score ( X ) = {\sum }_{i = 1}^{n \_ metrics} {w}_{i} {P}_{i} ( {x}_{i} )$
X represents the vector of metrics computed for a state. Pᵢ is a penalty function that computes the
error or penalty given a value of a metric and acceptable levels for that metric. For instance, this
penalty could be the absolute difference between the median of acceptable values of this metric and
the current instance value, or the squared difference, and so on. wᵢ is the weight associated with the
penalty for that metric, which is a scalar quantity. The weights w allow the scoring function to
weight different metrics more or less. For example, one scoring function might focus on overall
correctness, while another would focus on tooth alignment.
```

The method for this option can use metrics and/or constraints to customize final setups. For each treatment option, the method can either modify the optimal value for the metric, (change the penalty term (Pᵢ) in the Scoring function), or modify the constraints (change the Constrain function), or do both simultaneously, to achieve the desired outcome. For example:

Treatment duration—Constraints: increase limits on how much teeth are allowed to move during treatment. The Constrain function would move the teeth in the current state to a position in which the movement between the maloccluded state and the current state is less than a certain amount.

Treatment strategy, e.g., expansion:

Constraints: require minimal amount of tooth movement normal to the arch form to be greater than a threshold amount. The Constrain function would move the teeth in the current state to a position in which the expansion amount between the maloccluded state and the current state is no less than a certain amount.

Metrics: penalize movements that are less than a threshold amount. The penalty term in the scoring function would measure how much less the current movement is than the ideal amount of expansion.

Aligner features, e.g., attachments—Metrics: to minimize the number of attachments that are necessary, penalize certain types of tooth movement that would require attachments to be placed. The penalty term in the scoring function would measure the amount of tooth movement for certain types of movement (e.g., root torque).

### 3. Machine Learning-Based Approach (Step 44).

Treatment plans from previously treated patients are collected. These plans are separated into groups according to specific characteristics, for example:

Group 1: Cases with expansion between malocclusion and setup.

Group 2: Cases without expansion between malocclusion and setup.

Group 3: Cases with IPR in the setup.

Group 4: Cases with no IPR in the setup.

Group 5: Cases with fewer than 15 stages.

Group 6: Cases with greater than 15 stages.

Group 7: Cases with movement between malocclusion and setup for all tooth types.

Group 8: Cases with movement between malocclusion and setup for anterior teeth only.

Group 9: Cases planned by one member of a group of doctors.

From each of these (non-exhaustive) groups of cases, a different machine learning model is developed. When a new case is received by the system, each of these unique machine learning models can be applied to generate a treatment plan for each of these groups.

An example of a machine learning model is disclosed in co-pending Provisional Patent Application entitled, “System to Generate Staged Orthodontic Aligner Treatment,” and filed on even if fully set forth.

### 4. User-Specific Preferences (Step 46).

Preferred treatment strategies for specific users can also be learned by the system using one of several approaches identified below. These specific users can include, for example, doctors, technicians, patients, or others. Cases that adhere to specific preferred treatment methodologies can then be displayed as one or several of the options for treatment plans.

User feedback: Obtain preferred treatment strategies from users (e.g., doctors, technicians, or patients), or receive a completed form with their preferences.

Data analytics: Analyze cases from a specific user (e.g., doctor) and derive patterns.

Learn from data: Provide the user an exercise in which multiple setups for the same case are presented, and have the user either rank the setups or select which setup is preferred. Use this exercise to train a model to predict one or more of a set of setups that are most preferable to this user, and display these setups to that user.

Step 1: User (e.g, doctor or technician) has submitted a case and is offered the opportunity to participate in the exercise.

Step 2: User is instructed to select which setup they prefer.

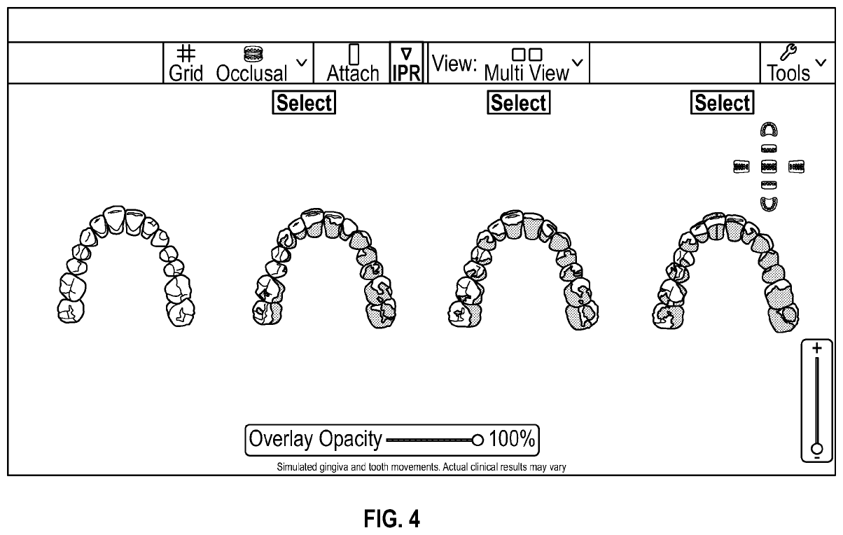

Step 3: User is shown a malocclusion and three versions of a setup and can select which version they prefer. This step may be repeated multiple times with different cases. FIG. 4 is a user interface to display the malocclusion (left image) and versions of a setup for treatment (second, third, and fourth images from the left).

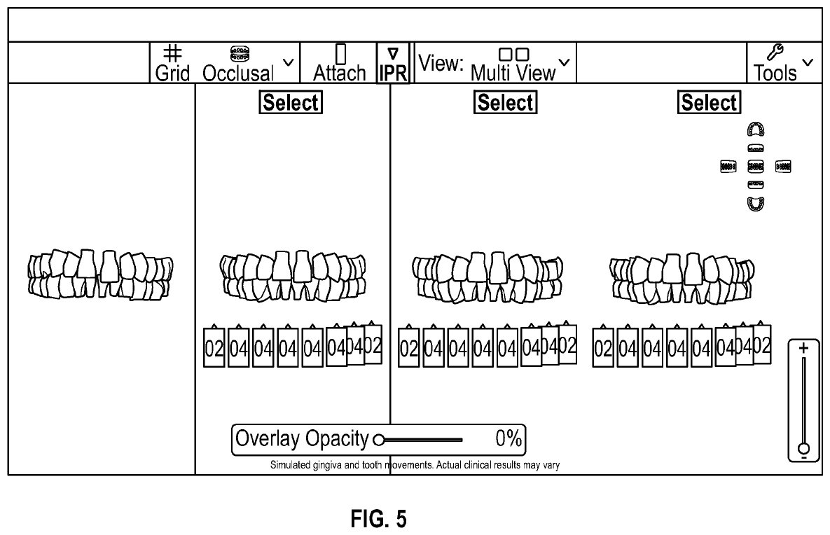

Step 4: For a new case that has been submitted, the user (e.g., doctor or technician) is shown multiple treatment options with the option that is best customized to their preferences highlighted. FIG. 5 is a user interface to display a malocclusion and multiple treatment options customized for particular preferences, for example showing the second image from left highlighted as best customized to the preferences.

The user interfaces in FIGS. 4 and 5 can be displayed on electronic display device 16 and include the opacity, zoom, and view tools as described with respect to FIG. 3. In FIG. 4, the shading on the digital 3D models of teeth represents the malocclusion overlays. In FIG. 5, the numbers below the digital 3D models of teeth represent the amount of IPR for each corresponding tooth.

The user interfaces in FIGS. 4 and 5 show three treatment plans for exemplary purposes; more or fewer treatment plans can be generated and shown in the same or a similar manner.

1. A computer-implemented method for generating multiple orthodontic treatment options, the method comprising:
receiving a digital 3D model of teeth in malocclusion;
generating a plurality of different orthodontic treatment plans for the teeth; and
displaying a visual indication of each of the plurality of different orthodontic treatment plans.

2. The method of claim 1, wherein generating the plurality of different orthodontic treatment plans comprises generating the plurality of different orthodontic treatment plans based upon rules for movement of the teeth relating to the malocclusion.

3. The method of claim 1, wherein generating the plurality of different orthodontic treatment plans comprises generating the plurality of different orthodontic treatment plans based upon metrics, constraints, or both metrics and constraints relating to movement of the teeth.

4. The method of claim 1, wherein generating the plurality of different orthodontic treatment plans comprises generating the plurality of different orthodontic treatment plans based upon treatment plans from previously treated patients.

5. The method of claim 1, wherein generating the plurality of different orthodontic treatment plans comprises generating the plurality of different orthodontic treatment plans based upon user-specific preferences.

6. The method of claim 1, wherein generating the plurality of different orthodontic treatment plans comprises generating one of the orthodontic treatment plans to include aligners.

7. The method of claim 1, wherein generating the plurality of different orthodontic treatment plans comprises generating one of the orthodontic treatment plans to include brackets.

8. The method of claim 1, wherein generating the plurality of different orthodontic treatment plans comprises generating one of the orthodontic treatment plans to include a combination of aligners and brackets.

9. The method of claim 1, wherein generating the plurality of different orthodontic treatment plans comprises generating one of the orthodontic treatment plans based upon a treatment duration.

10. The method of claim 1, wherein generating the plurality of different orthodontic treatment plans comprises generating one of the orthodontic treatment plans based upon a treatment finishing approach.

11. The method of claim 1, wherein generating the plurality of different orthodontic treatment plans comprises generating one of the orthodontic treatment plans based upon a treatment intervention.

12. The method of claim 1, further comprising receiving a user selection of one of the plurality of different orthodontic treatment plans.

13. The method of claim 12, further comprising manufacturing appliances for the user selected orthodontic treatment plan.

14. The method of claim 1, further comprising receiving a user refinement of one of the plurality of different orthodontic treatment plans.

15. The method of claim 1, further comprising receiving a user request for refinement of one of the plurality of different orthodontic treatment plans.

16. A system for generating multiple orthodontic treatment options, the system comprising a computing device comprising:
an interface configured to receive a digital 3D model of teeth in malocclusion;
processing circuitry configured to generate a plurality of different orthodontic treatment plans for the teeth; and
a display device in communication with the processing circuitry, the display device being configured to display a visual indication of each of the plurality of different orthodontic treatment plans.

17. A computer-implemented method for displaying a user interface comprising multiple orthodontic treatment options for a digital 3D model of teeth in malocclusion on an electronic display device, the method comprising:
displaying, as part of the user interface, a visual indication of each of a plurality of different orthodontic treatment plans for the digital 3D model of teeth, wherein the visual indication includes one or more of:
the digital 3D model of teeth in malocclusion shown separately with overlays between the malocclusion and a final state of the teeth for the treatment plans,
only the malocclusion overlays of the treatment plans without the digital 3D model of teeth in malocclusion shown separately,
the digital 3D model of teeth in malocclusion shown separately with the treatment plans and without the malocclusion overlays, or
the treatment plans without the digital 3D model of teeth in malocclusion shown separately and without the malocclusion overlays.

18. The method of claim 17, wherein one of the displayed orthodontic treatment plans includes aligners.

19. The method of claim 17, wherein one of the displayed orthodontic treatment plans includes brackets.

20. The method of claim 17, wherein one of the displayed orthodontic treatment plans includes a combination of aligners and brackets.

21. (canceled)
